(19) United States
(12) Patent Application Publication
(10) Pub. No.: US 2016/0053026 A1
(43) Pub. Date: Feb. 25, 2016

(54) METHODS FOR TREATING INFECTION BY HPV

(71) Applicant: STC.UNM, ALBUQUERQUE, NM (US)

(72) Inventors: MICHELLE A. OZBUN, ALBUQUERQUE, NM (US); JULIE E. BAUMAN, ALBUQUERQUE, NM (US)

(21) Appl. No.: 14/877,191

(22) Filed: Oct. 7, 2015

Related U.S. Application Data
(63) Continuation of application No. 13/455,665, filed on Apr. 25, 2012.
(60) Provisional application No. 61/478,648, filed on Apr. 25, 2011.

(51) Int. Cl. C07K 16/40; A61K 31/366; A61K 31/519; A61K 31/5377; A61K 31/517; A61K 31/352

(52) U.S. Cl. CPC C07K 16/40; A61K 31/517; A61K 31/352; A61K 31/519; A61K 31/5377; A61K 31/366; C07K 2317/24; C07K 2317/76

(57) ABSTRACT

We describe herein methods for treating HPV infections and medical conditions caused by HPV infections. Generally, the methods include administering to a subject exhibiting at least one symptom or clinical sign of HPV infection a composition that includes an EGFR signaling inhibitor in an amount effective to ameliorate the at least one symptom or clinical sign of HPV infection.

### CROSS-REFERENCE TO RELATED APPLICATION

This application claims priority to U.S. Provisional Patent Application Ser. No. 61/478,648, filed Apr. 25, 2011, the entirety of which is incorporated by reference herein.

### BACKGROUND

Epidermal Growth Factor Receptor (EGFR) is a 180 kDa transmembrane glycoprotein composed of an intracellular tyrosine kinase domain and an extracellular ligand-binding domain. EGFR binding of ligands (e.g., EGF, TGFα) can activate a downstream signaling cascade that can be involved in cell survival, proliferation, and other vital cellular functions. EGFR and has been implicated in oncogensis and/or angiogenesis in some cancer cells (Hu, et al., 1997, J Natl Cancer Inst 89:1243-1246).

Mutations that lead to EGFR overexpression or increased EGFR activity have been associated with a number of cancers, including lung cancer, anal cancers, and glioblastoma multiforme. Mutations, amplifications, or misregulations of EGFR or family members are implicated in about 30% of all epithelial cancers. Mutations involving EGFR can lead to its constant activation, which can result in uncontrolled cell division—a predisposition for cancer. Consequently, mutations of EGFR have been identified in several types of cancer, and it is the target of an expanding class of anticancer therapies.

Anticancer therapeutics directed against EGFR include, for example, gefitinib and erlotinib for lung cancer, cetuximab for colon cancer, panitumumab for colorectal cancer, zalutumumab for squamous cell carcinoma of the head and neck (SCCHN), nimotuzumab for SCCHN, malignant astrocytoma, and glioma, and matuzumab for colorectal, lung, esophageal, and stomach cancer. Certain of these therapeutics are monoclonal antibodies that block the extracellular ligand binding domain of EGFR, thereby inhibiting the binding of a ligand with EGFR and the resulting activation of the downstream signaling cascade. Examples of therapeutic monoclonal antibodies include cetuximab, panitumumab, zalutumumab, nimotuzumab, and matuzumab. Other EGFR therapeutics include kinase inhibitors that target the cytoplasmic side of the receptor and are designed to inhibit the ability of EGFR, upon binding of a ligand, to initiate the downstream signaling pathway. Gefitinib, erlotinib, and lapatinib (mixed EGFR and ERBB2 inhibitor) are examples of small molecule kinase inhibitors. In addition, there are a number of inhibitors that target downstream effects of EGFR signaling and the targets PKC, Ras/MAPK, and AKT/mTOR.

### SUMMARY OF THE INVENTION

This disclosure describes a method of treating an infection by human papillomas virus (HPV). Generally, the method includes administering to a subject exhibiting at least one symptom or clinical sign of HPV infection a composition that includes an EGFR signaling inhibitor in an amount effective to ameliorate at least the exhibiting at least one symptom or clinical sign of HPV infection.

In some cases, the at least one symptom or clinical sign of HPV infection can include at least one symptom or clinical sign of a cancer such as, for example, cervical cancer or oral cancer. In other embodiments, the at least one symptom or clinincal sign of HPV infection can include respiratory papillomatosis, cervical dysplasia, the appearance of warts, or the appearance of genital warts.

In some cases, the EGFR signaling inhibitor comprises an antagonist of EGFR. In some of these embodiments, the antagonist of EGFR can include a monoclonal antibody. In other embodiments, the EGFR signaling inhibitor can include a tyrosine kinase inhibitor. In still other embodiments, the EGFR inhibitor can include an inhibitor of MAPK, an inhibitor of AKT, or an inhibitor of PKC.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Model for the effects of HPV infection and gene expression on the ERB/Receptor Tyrosine Kinase (RTK) family/and downstream signaling pathways.

FIG. 2. Model for the activities of ERB or RTK signaling inhibitors on HPV functions in HPV infected cells.

FIG. 3A-3B. Total EGFR levels and response to EGF stimulation in SG3 (NIKS cells maintaining episomal HPV16; HPV[+]) and NIKS HPV[−] HFK cells. Cells were incubated in SFM over night before exposure to 10 ng/mL EGF for indicated times before harvesting and subjecting to SDS-PAGE and immunoblot for EGFR and GAPDH, a loading control (A). Quantification of EGFR levels normalized to GAPDH levels (B). Results are representative of three independent experiments.

FIG. 4. Relative infection levels following exposure of cells to HPV16 in the presence of cetuximab, or small molecule inhibitors of EGFR (AG1478, PD168393), or the tyrosine kinase inhibitor genistein.

FIG. 5. Response of Erb RTK pathway signaling in HPV[−] or HPV[+] human keratinocytes following inhibition of EGFR with cetuximab and MEK with PD98059. Cells were serum starved for 8 hours prior to mock (M) treatment, treatment with diluent in SFM and 10 ng/ml EGF (Ø), cetuximab (C, 100 μg/mL) or MEK inhibitor PD98059 (P, 25 μM) before and during exposure to 10 ng/mL EGF in SFM for indicated times. Mock=No EGF. Cell lysates were harvested and subjected to SDS-PAGE and immunoblot for p-EGFR, total EGFR, p-ERK1/2 and GAPDH, a loading control.

FIG. 6A-6C. The ATK/mTOR pathway is activated and PTEN is inhibited in response to HPV infection. Human keratinocytes were serum-starved for 4 hours, then mock exposed or exposed to EGF (5 ng/mL), HPV16 (100 vge/cell) for the times indicated. (A). After serum-starvation, cells were treated with an inhibitor (1 μM AG1478 [EGFR inhibitor], 25 μM LY294002 [PI3K inhibitor] or 1 nμM Wortmannin [PI3K inhibitor]) or DMSO for one hour at 37° C. Following incubation with inhibitors, cells were treated with 100 vge/cell HPV16 PsV for 15 minutes at 37° C. in the presence of inhibitors. Cells werebwashed with ice cold PBS and lysed with RIPA buffer, clarified, solubilized in loading buffer and fractionated by SDS-PAGE. Immunoblot analysis was performed using anti-pAKT (Ser 473) and pAKT (Thr 308) antibodies. Data are representative of three independent assays. (B) The cell lysates were analyzed by SDS-PAGE and immunoblot for p-PTEN (Ser 380), an inhibitor of ATK/mTOR. (C) Cell lysates were analyzed for p-mTOR (Ser 2448 and Ser 2481). Each was phosphorylated in response to HPV exposure.

FIG. 7. Effect of Erb and MAPK inhibitors on cells survival in HPV[−] and HPV[+] human keratinocytes. Cells were grown for seven days in fibroblast conditioned media containing 0 μg/mL [M], 50 μg/mL, 100 μg/mL, or 200 μg/mL Cetuximab or 12.5 μM, 25 μM, or 50 μM PD98059as indicated by the wedges. The boxes indicate the same concentrations used in the FIG. 3 and FIG. 5. MTT assays were performed to assess cell viability. Error bars represent SEM (N=3).

FIG. 8. HPV16 E1̂E4 early mRNA levels in response to treatment with EGFR or MAPK inhibitors. HPV[+] HFK cells incubated in fibroblast conditioned media containing 100 μg/mL Cetuximab or 25 μM PD98059. Total RNA harvested at indicated time points was subjected to RT and then to qPCR for β-actin and HPV16 E1̂E4 quantification. Observed decrease in HPV16 E1̂E4 transcript levels in response to both treatments and decrease is statistically significant after 48 hours of treatment but levels appear to rise after this time point. Data are the result of two separate experiments.

FIG. 9. Response of HPV16 viral genome number to EGFR or MAPK Inhibitors. HPV[+] HFK cells incubated in fibroblast conditioned media containing 100 μg/mL Cetuximab (M=Media only) or 25 μM PD98059 (Ø=Media+DMSO). Total DNA harvested at indicated time points was subjected to qPCR for HPV16 LCR quantification. Observed statistically significant decrease in HPV16 genome levels as compared to mock treated in each group in response to both treatments. Data are the result of 2 separate experiments. (* p<0.05, ** p<0.01).

### DETAILED DESCRIPTION OF ILLUSTRATIVE EMBODIMENTS

We describe herein methods for treating HPV infections and medical conditions caused by HPV infections. Generally, the methods include administering to a subject exhibiting at least one symptom or clinical sign of HPV infection a composition that includes an EGFR signaling inhibitor in an amount effective to ameliorate at least the exhibiting at least one symptom or clinical sign of HPV infection.

As used herein, the following terms shall have the indicated meanings

“Ameliorate” refers to any reduction in the extent, severity, frequency, and/or likelihood of a symptom or clinical sign characteristic of a particular condition.

“Co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co- administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

“EGFR signaling inhibitor” and variations thereof shall refer to a compound or composition that reduces EGFR-dependent signaling, regardless of whether the compound or composition directly binds to EGFR, a ligand of EGFR, or a downstream target of the EGFR pathway. An EGFR inhibitor may be, for example, an EGFR antagonist, a compound or composition that inhibits the EGFR tyrosine kinase domain, and/or interferes with AKT-induced and/or MAPK-induced expression.

“EGFR antagonist” and variations thereof refer to a compound or composition that binds to the extracellular domain or the intracytoplasmic tyrosine kinase domain of EGFR and results in a level of EGFR cell signaling that is less than the EGFR-dependent cell signaling induced by any natural agonist of EGFR.

“Express” and variations thereof refer to the ability of a cell to transcribe a coding region of a polynucleotide sequence, resulting in an mRNA, then translating the mRNA to form a protein that provides a detectable biological function to the cell.

“Induce” and variations thereof refer to any measurable increase in cellular activity. For example, induction of a particular cytokine refers to an increase in the production of the cytokine As another example, induction of a nucleotide sequence refers to an increase in transcription of (for, e.g., a coding sequence) or from (for, e.g., a regulatory sequence such as a promoter) the nucleotide sequence.

“Inhibit” and variations thereof refer to any measurable reduction of cellular activity. For example, inhibition of a particular cytokine refers to a decrease in production of the cytokine As another example, inhibition of a nucleotide sequence refers to a decrease in transcription of (for, e.g., a coding sequence) or from (for, e.g., a regulatory sequence such as a promoter) the nucleotide sequence. The extent of inhibition may be characterized as a percentage of a normal level of activity.

“Ligand” and variations thereof refer to a compound that is capable of binding to another, specified compound (e.g. a molecule capable of binding to a receptor).

“Subject” includes, for example, animals such as, but not limited to, humans, non-human primates, companion animals such as, for example, dogs, cats, birds, or rodents; and livestock animals such as, for example, horses, pigs, sheep, goats, or cows.

“Sign” or “clinical sign” refers to an objective physical finding relating to a particular condition capable of being found by one other than the patient.

“Specific” and variations thereof refer to having a differential or a non-general (i.e., non-specific) affinity, to any degree, for a particular target.

“Symptom” refers to any subjective evidence of disease or of a patient's condition.

“Therapeutic” and variations thereof refer to a treatment that ameliorates one or more existing symptoms or clinical signs associated with a condition.

“Treat” or variations thereof refer to reducing, ameliorating, or resolving, to any extent, the symptoms or signs related to a condition.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

We have discovered that EGFR-MAPK cell survival functions and HPV genome transcription can be involved in a positive feedback loop. EGFR-MAPK signaling and HPV gene expression, however, do not necessarily require EGFR overexpression. Consequently, HPV infection has not been viewed as an indication treatable with a therapeutic EGFR inhibitor. EGFR inhibitors can reduce—and in some cases even eliminate—MAPK signaling, thereby reducing viral oncoprotein transcription and enhancing and/or restoring tumor suppressor p53 and pRb proteins.

In response to an EGFR inhibitor such as, for example, cetuximab, the MAPK signal cascade and AP-1 are not activated, lowering transcription of HPV oncoproteins. Therefore, cetuximab may function as an antiviral in this case.

HPV gene expression and the splicing of mRNA that encodes various HPV oncoproteins are regulated by the EGF pathway. Three viral oncoproteins, E5, E6 and E7, either enhance EGFR activation (E5, and possibly E6, E7) or inhibit tumor suppressor proteins p53 and pRb (E6, E7). The HPV gene enhancer contains AP1 binding sites c-fos and c-jun, which are also part of the DNA proliferation cell mechanism. Thus, there may be cooperation between HPV oncoprotein expression and the EGFR-MAPK pathway that promotes cell proliferation. Therefore, EFGR inhibitors can reduce AP-1 transcription factor activation and, therefore, reduce HPV transcription, viral genome replication and cell proliferation.

Epidermal growth factor receptor (EGFR), also sometimes referred to as Her 1 or ErbBl, is involved in the development of the EGF-dependent cancer squamous cell carcinoma (SCC) and signals through the Ras-MAPK, PI3K-PTEN-AKT and phospholipase C pathways (Hynes et al., 2009, Curr Opin Cell Biol 21:177-184). EGFR activation of Ras can initiate a multistep phosphorylation cascade that can lead to the activation of MAPKs including ERK1/2 (Hynes et al., 2009, Curr Opin Cell Biol 21:177-184). Subsequently, ERK1/2 can regulate cell transcription via c-fos/c-jun and/or many other transcription factors involved in cell proliferation, survival, and transformation in vitro (Lewis et al., 1998, Adv Cancer Res 74:49-139). Increased MAPK activation has been reported for several human tumors. Deregulated EGFR-MAPK signaling can confer cancer cell survival and can enhance resistance to chemotherapy. Therefore, EGFR pathway inhibitors have been developed to act as potential molecular targeting drugs for treating EGFR-dependent cancers.

The anticancer therapeutics directed against EGFR include gefitinib (Paez et al., 2004, Science 304:1497-1500) and erlotinib for lung cancer, cetuximab for colon cancer and head and neck cancer, and lapatinib (also known as GW583340) for breast cancer and other solid tumors. The monoclonal antibody cetuximab can bind to the extracellular domain of EGFR, and thereby can block binding of EGF to EGFR. Cetuximab can, therefore, inhibit receptor phosphorylation, inhibit receptor activation, promote EGFR internalization, promote EGFR degradation, and/or lead to EGFR downregulation (Vincenzi et al., 2008, Critical Reviews in Oncology/Hematology 68:93-106). Thus, signal transduction that promotes proliferative effects can be blocked by cetuximab. Cetuximab also can facilitate antibody-dependent cellular cytotoxicity that can contribute to anti-tumor effects (Vincenzi et al., 2008, Critical Reviews in Oncology/Hematology 68:93-106).

Gefitinib, the first selective inhibitor of EGFR tyrosine kinase domain, and erlotinib target EGFR tyrosine kinase function by binding reversibly to the ATP binding site of the receptor (Raymond et al., 2000, Drugs 60 Suppl 1:15-23). Lapatinib can inhibit the tyrosine kinase activities associated with ErbB1 and ErbB2 (Her2). Thus, the tyrosine kinase inhibitors generally function to block the ability of EGFR tyrosine kinase to activate the anti-apoptotic Ras signal transduction cascade. As a result, malignant cell proliferation can be restrained.

A number of other drugs have been shown to reduce signaling from EGFR and/or more distant kinases in the EGFR-MAPK pathway. Tyrphostin AG1478 is a potent and specific drug inhibitor of EGFR tyrosine kinase. PD168393 is a potent, cell-permeable, irreversible, and selective inhibitor of EGF receptor (EGFR) tyrosine kinase activity. GW572016 suppresses the activation of EGFR, ErbB2, MAPK, and AKT in a concentration-dependent manner (Zhou et al., 2006, Cancer Research 66:404-411). PD98059 is a highly selective inhibitor of MAPK (ERK1/2). U0126 and SL327 are dual MEK1 & MEK2 inhibitors. Other drugs that can be suitable EGFR signaling inhibitors include, for example, farnesyl transferase inhibitor R115777 (tipifarnib, e.g., ZARNESTRA, Johnson & Johnson Research & Development, LLC, Raritan, N.J.), the Raf antisense oligonucleotide ISIS 5132, the Raf inhibitor Bay 43-9006 (sorafinib, e.g., NEXAVAR, Onyx Pharmaceuticasls, Inc., South San Francisco, Calif. and Bayer HealthCare Pharmaceuticals, Inc., Berlin, Germany), the MEK inhibitor CI-1040 (PD184352, Pfizer, Inc., New York, NY), the mTOR inhibitor CCI-779 (temsirolimus, e.g., TORISEL, Wyeth Pharmaceuticals, Collegeville, Pa.), the mTOR inhibitor RAD001 (everlimus, e.g., ZORTRESS,

Novartis Pharmaceuticals Corp., East Hanover, N.J.), the topoisomerase II inhibitor C1311 (e.g., SYMADEX, Xanthus Pharmaceuticals, Inc., Cambridge, Mass.), and the heat shock protein 90 (Hsp90) inhibitor tanespimycin (Bristol-Myers Squibb, New York, N.Y.).

Human papillomaviruses (HPVs) are a group of more than 150 related viruses. They are called papillomaviruses because certain types may cause warts, or papillomas, which are benign (i.e., noncancerous) tumors. Some types of HPV, however, are associated with certain types of cancer. These are called “high-risk,” oncogenic, or carcinogenic HPVs. Other sexually transmitted types of HPV do not appear to cause cancer and are called low-risk HPVs.

Although genital HPV infections are very common, most occur without any symptoms and go away without any treatment within a few years. However, some HPV infections can persist for many years. Persistent infections with high-risk HPV types can cause cell abnormalities. If untreated, areas of abnormal cells, called lesions, can sometimes develop into cancer. Persistent HPV infections are now recognized as the cause of most cases of cervical cancer. Cervical cancer is diagnosed in nearly half a million women each year worldwide, claiming a quarter of a million lives annually. Almost all women will have an HPV infection at some point, but very few will develop cervical cancer. The immune system of most women will usually suppress or eliminate HPVs. Typically, only HPV infections that are persistent (i.e., HPV infections that do not go away over many years) can lead to cervical cancer. High-risk HPVs also may be involved in the development of some cancers of the anus, vulva, vagina, and penis. In addition, oral HPV infection causes some cancers of the oropharynx, i.e., the middle part of the throat including, for example, the soft palate, the base of the tongue, and the tonsils. In total, it has been estimated that HPV infection accounts for approximately five to seven percent of all cancers worldwide.

Both high-risk and low-risk types of HPV can cause the growth of abnormal cells. For example, HPV types 1 and 2 can cause common and plantar warts, and HPV6 and HPV11 can cause recurrent respiratory papillomatosis and genital warts; these lesions can have significant associated morbidity. Only the high-risk types of HPV, however, lead to cancer. About 15 high-risk HPV types have been identified, including HPV types 16 and 18, which together cause about 70 percent of cervical cancers. HPV16 is the predominant HPV genotype associated with HPV-related oropharyngeal cancers.

Although there is currently no therapeutic medical treatment for HPV infections, the cervical lesions and warts that can result from such infections can be treated. Methods commonly used to treat cervical lesions include cryosurgery (i.e., freezing that destroys tissue), loop electrosurgical excision procedure (LEEP), and conization (i.e., surgery to remove a cone-shaped piece of tissue from the cervix and cervical canal). Similar treatments may be used for external genital warts. Surgical debridement is commonly used to treat respiratory papillomatosis.

Prophylactic treatments for HPV infection can include HPV vaccines such as GARDISIL (Merck & Co., Inc., Whitehouse Station, N.J.) and CERVARIX (GlaxoSmithKline, Philadelphia, Pa.). The GARDASIL vaccine is a quadrivalent vaccine that protects against four HPV types: 6, 11, 16, and 18. The FDA has approved GARDASIL for use in females for the prevention of cancers caused by HPV types 16 and/or 18 including, for example, cervical cancer and some vulvar and vaginal cancers. GARDICIL also has been approved for use in males and females for the prevention of genital warts caused by HPV types 6 and/or 11. CERVARIX is a bivalent vaccine that targets two HPV types: 16 and/or 18. The FDA has approved CERVARIX for use in females ages 10 to 25 for the prevention of cervical cancer caused by HPV types 16 and/or 18.

Neither of these HPV vaccines has been proven to provide complete protection against persistent infection with other HPV types, although some initial results suggest that either vaccine might provide partial protection against a few additional HPV types that can cause cervical cancer. Overall, therefore, even with complete vaccine uptake, about 30 percent of cervical cancers will not be prevented by these vaccines. Also, in the case of GARDASIL, about 10 percent of genital warts will not be prevented by the vaccine.

Although these vaccines can help prevent HPV infection, they do not help eliminate existing HPV infections and cannot be used to treat, for example, genital warts, oral lesions or cervical cancer. For example, one recent study found that CERVARIX was not effective in helping women who are already infected to clear the infection (Hildesheim et al., 1989, JAMA 298(7):743-753).

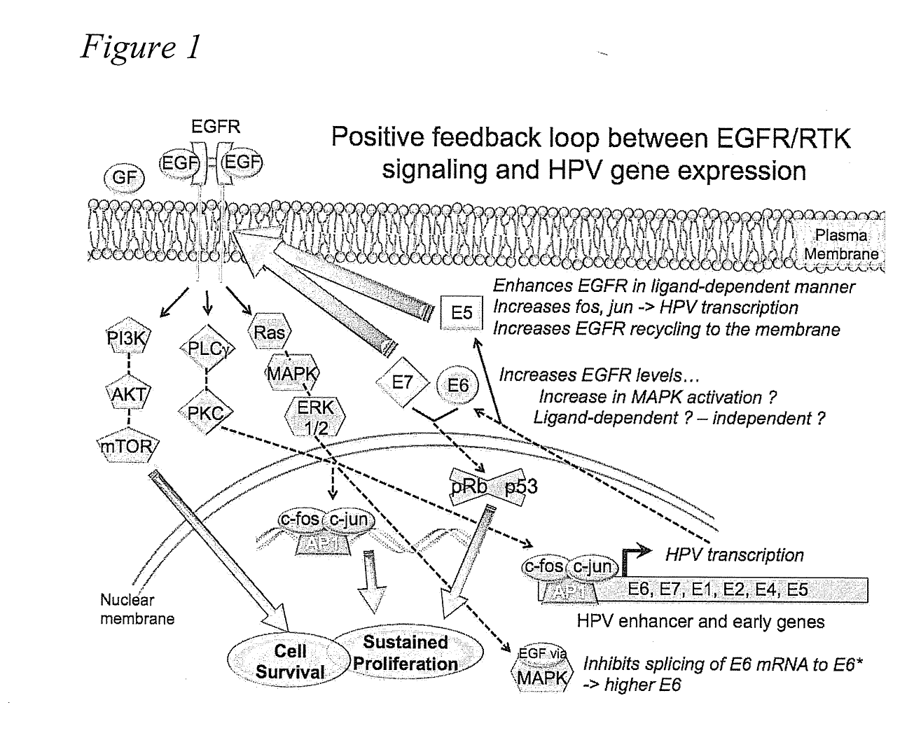

A model for the effects of HPV infection and gene expression on the ERB/Receptor Tyrosine Kinase (RTK) family/MAPK pathway is shown in FIG. 1. Ligand (e.g., EGF, or other growth factor [GF]) binding can activate signaling through EGFR/RTK and can trigger one, two or all three of the AKT, PKC, and MAPK pathways.

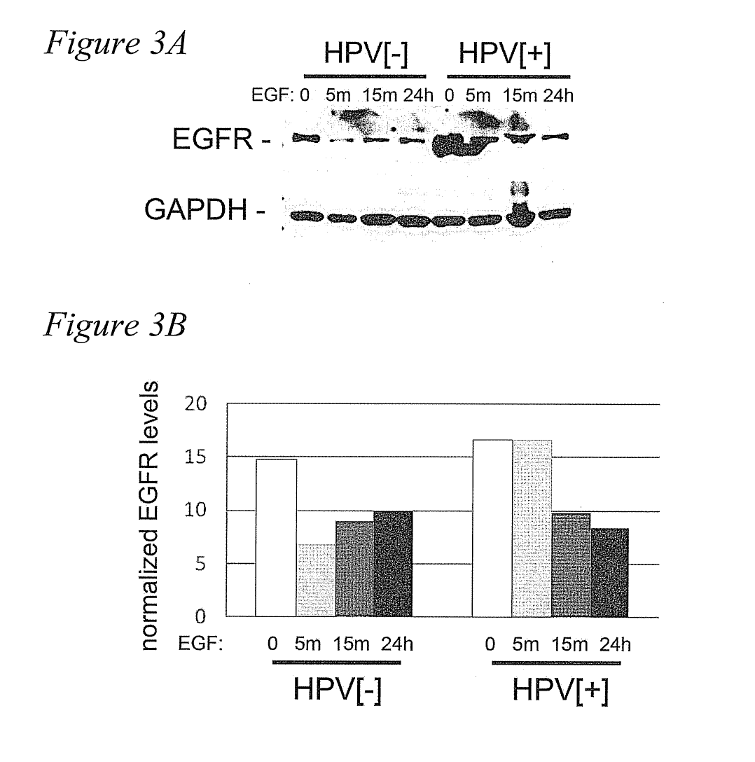

Carcinogenic HPVs encode three oncoproteins. The E5 protein has long been recognized to enhance EGFR activation in a ligand-dependent manner (Crosius et al., 1998, Experimental Cell Research 241:76-83), making cells more sensitive to lower EGF concentrations (Pim et al., 1992, Oncogene 7:27-32). E6 and E7 are multifunctional proteins capable of functionally inhibiting tumor suppressor proteins p53 and pRb, respectively (zur Hausen et al., 1989, Adv. Viral Onc. 8:1-26). Additional findings are consistent with a role for EGFR-MAPK signaling in HPV infections and related tumorigenesis (FIG. 1), but these have been largely overlooked as neoplastic mechanisms, primarily because EGFR levels on the plasma membrane are not substantively higher in HPV infected cells or cervical cancers (see FIG. 3). Inhibiting E6 and E7 expression in tumor cells can reduce both EGFR protein levels and cell proliferation (Hu et al., 1997, J Natl Cancer Inst 89:1243-1246). The EGF pathway can regulate HPV gene expression and splicing of E6-E7 mRNA: the viral enhancer contains AP1 (c-fos, c-jun) transcription factor binding sites and these proteins can increase HPV early transcription including, for example, transcription of mRNAs encoding E6, E7, E1, E2, E4, ES proteins. E6-E7 mRNA is EGF-inducible in HPV16[+] SiHa cells (Peto et al., 1995, Journal of General Virology 76:1945-1958). The presence of EGF can favor E6 expression, whereas EGF depletion and/or EGFR inhibition can favors mRNA splicing of E6*, which can result in increased p53 levels and enhanced translation of E7 (Rosenberger et al., 2010, Proceedings of the National Academy of Sciences 107:7006-7011). Consistent with a role for E5 in enhancing EGFR activation (Crosius et al., 1998, Experimental Cell Research 241:76-83), E5-expressing cells can have higher c-fos and c-jun levels and more active HPV 16 transcription. Further, expression of E5 and E7 in primary cells can produce a potent mitogenic response that can be enhanced by EGF. These observations suggest HPV oncoprotein expression can provide a positive feedback loop for the EGFR-MAPK pathway, maintaining cell proliferation (FIG. 1).

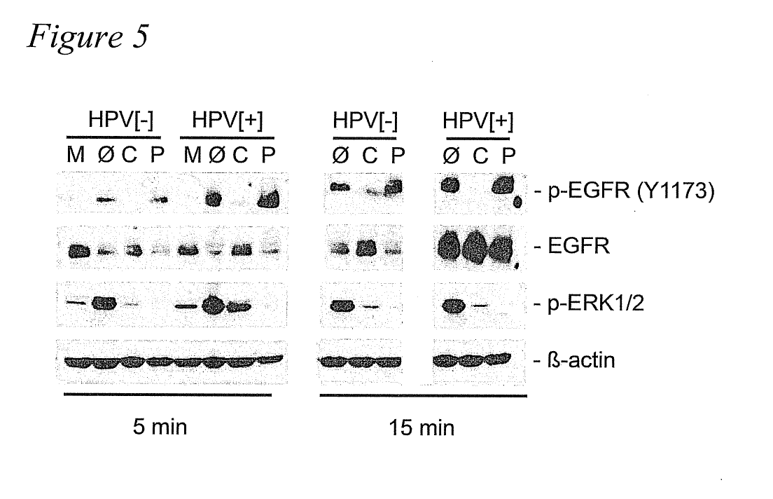

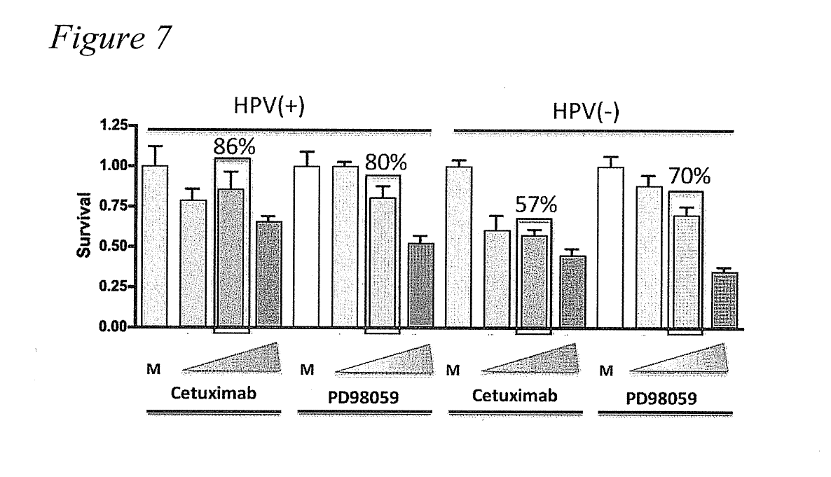

Cetuximab's effect was recently assessed on MAPK activation and proliferation in two cervical cancer cell lines long in culture, but with no investigation of HPV involvement (Meira et al., 2009, Br J Cancer 101:782-791). Our approach was similar, investigating differences between HPV16 positive (HPV[+]) and HPV negative (HPV[−]) cell lines, however, and investigating the effects of additional EGFR signaling inhibitors. Cells were grown in monolayer cultures and exposed to clinically relevant doses of EGF (0.1 ng/ml or 10 ng/ml, 5-15 minutes), cetuximab (100 μg/ml, 4 hours), PD98059 (25 μM, 1 hour). Western blot was performed (FIG. 5) and cell viability determined with MTT assay and clonogenic assays performed as described (FIG. 7) (Meira et al., 2009, Euro J Cancer 45:1265-1273).

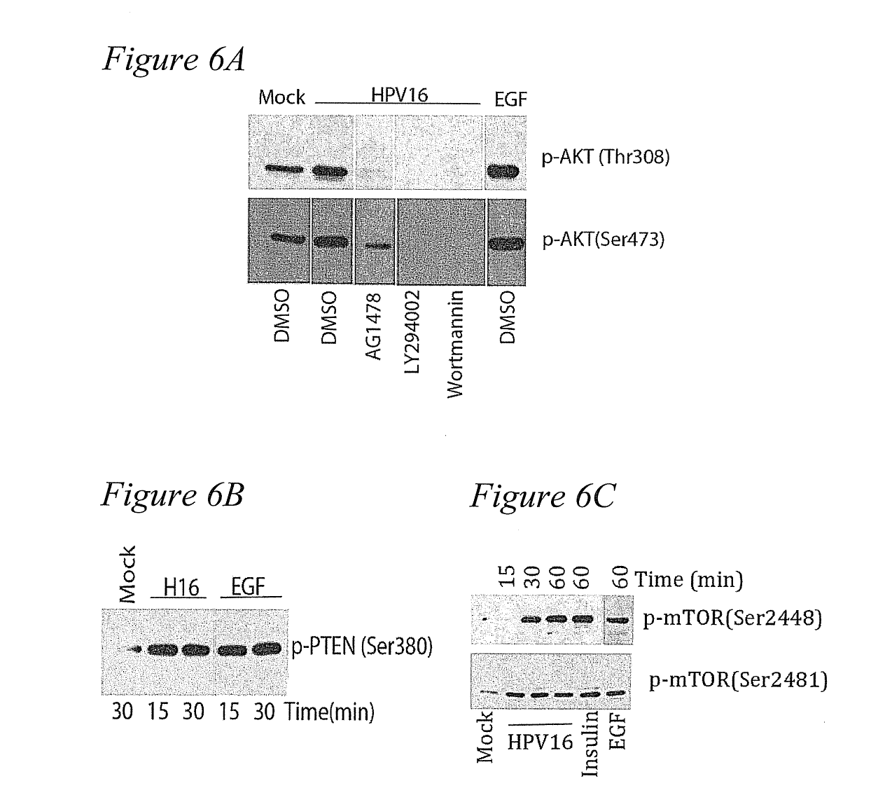

As shown in FIG. 1, HPV and Erb/MAPK interactions can create a positive feedback loop independent of the amount of EGFR in the plasma membrane. Basal levels of EGFR and levels following EGF activation were assessed (FIG. 3). HPV[+] and HPV[−] cells have similar total EGFR levels, but HPV[+] cells downregulate EGFR levels more slowly in response to EGF stimulation compared to HPV[−] cells (FIG. 3 and FIG. 6). Thus, EGFR signaling is more active in HPV[+] cells.

Treatment with cetuximab caused decreased EGFR activation and decreased downstream signaling (FIG. 5). The MEK inhibitor PD98059 did not affect EGFR activation, but blocked MAPK signaling. These inhibitors affected both cell lines. The HPV[−] cell line is more responsive to signal inhibitors, however, indicating that HPV16 activates these pathways.

The proliferative capacity of MAPK inhibitor-treated cells compared to untreated cells was measured by MTT assay after 72 hours to determine the surviving fractions (FIG. 7). A dose-dependent decrease in viability of the HPV[+] and the HPV[−] cells was seen when treated with the inhibitors cetuximab and PD98059.

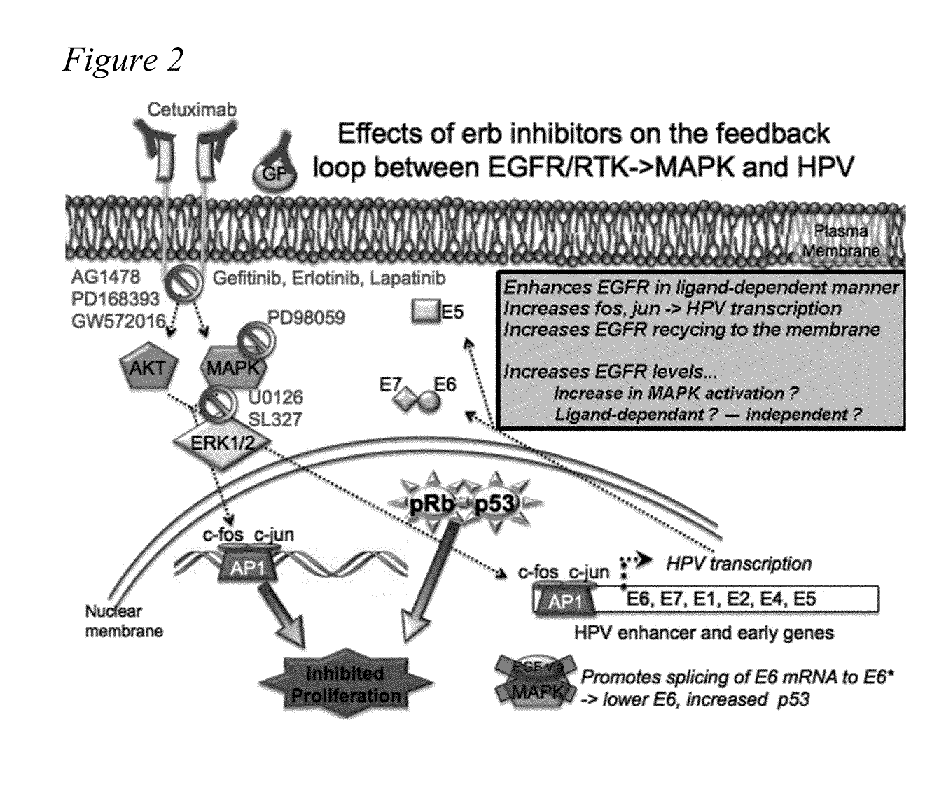

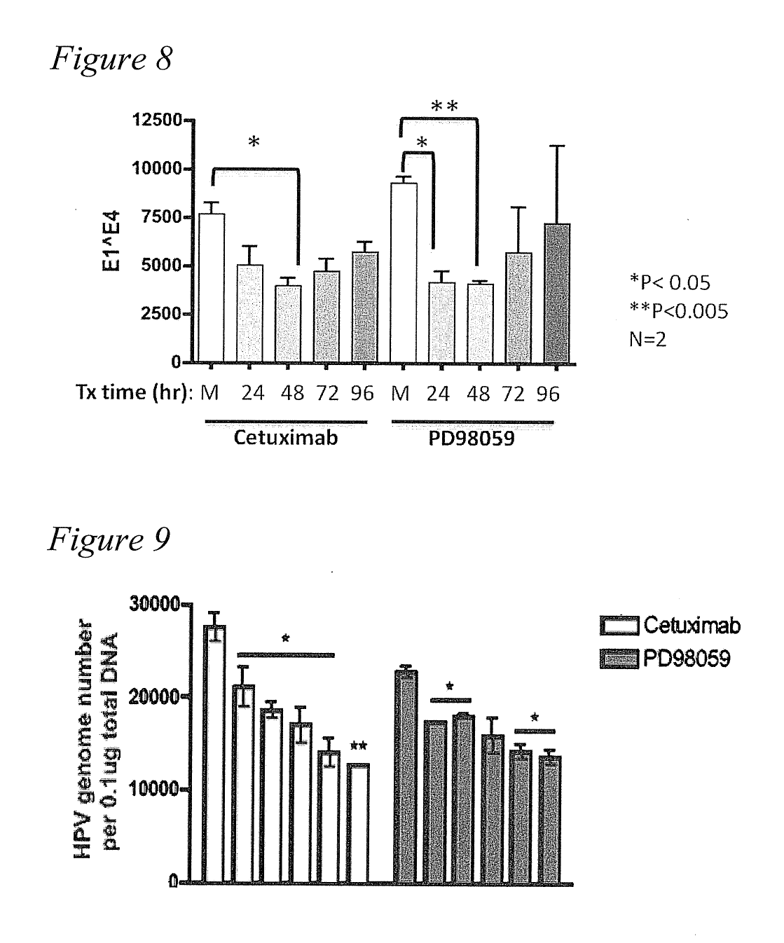

As illustrated in FIG. 2, inhibitors of receptor tyrosine kinase (RTK) and/or MAPK can reduce activity along signal pathways such that HPV transcription, including oncoprotein expression, is reduced. Reduced HPV expression can restore functional levels of p53 and pRb. We quantified HPV early mRNAs E1̂E4 /E5 using RT-qPCR following the exposure of the cell lines to the active doses of EGF, cetuximab, and PD98059 used in FIGS. 6 and FIG. 7. The results showed that both inhibitors reduced viral gene expression significantly (≦50% lower) by 24-48 hours post treatment (FIG. 8).

HPV early proteins cannot be detected by standard methods. Therefore, one can instead determine expression levels of surrogate markers of HPV E6 and E7 expression, p53 as a surrogate for E6 and pRb and/or p16 as surrogate markers for E7 (Rampias et al., 2009, J Natl Cancer Inst 101:412-423). E6 degrades p53, so if MAPK inhibits E6 expression, p53 levels will increase. Likewise, E7 inhibits pRb, resulting in increased p16 levels. Thus, MAPK inhibition of E7 can release pRb and downregulate p16.

The ATK pathway is activated and PTEN is inhibited in response to HPV infection (FIG. 6). Human keratinocytes exposed to HPV16 or HPV31 showed the same activation of the AKT arm of EGFR signaling as did simple treatment with the EGFR ligand, EGF. Therefore, inhibitors of this pathway can inhibit HPV infection as seen for the EGFR inhibitors.

This work provides a direct comparison of the biological effects of cetuximab and the MAPK inhibitor PD98059 on HPV[+] and HPV[−] cells. We determined that HPV status affects the biochemical and proliferative response of HPV[−] vs. HPV[+] cells to EGFR inhibition. HPV status also affects the influence of EGFR inhibition on HPV gene expression and potential feedback to the MAPK pathway. Understanding the response of HPV[+] cells to EGFR signaling inhibition stands to benefit patients with any type of HPV infection.

Therefore, EGFR inhibitors may have utility for treating HPV infections and, in particular, HPV[+] tumors such as, for example, cervical cancer, HPV[+] oral cancer, and HPV[+] head-and-neck cancers. A model for the activities of ERB or MAPK inhibitors on HPV functions in HPV infected cells is shown in FIG. 2. An EGFR signaling inhibitor—including, for example, an EGFR antagonist, a compound or composition that inhibits the EGFR tyrosine kinase domain, a MAPK inhibitor, an AKT inhibitor, and PKC inhibitor—can cause diminished HPV early gene expression due to its ability to reduce AP-1 transcription factor activation. Lower levels of the early gene products E5, E6, and/or E7 can further diminish EGFR signaling. Lower HPV early gene expression can reduce viral genome levels if the genome is episomal and this can result in near or complete viral clearance if the viral genome is not integrated. If the viral genome is integrated, the decreased viral protein expression also may result in restored p53 and pRb expression and allow the cells to activate the apoptotic pathway resulting in cell death. In this way, an EGFR signaling inhibitor can act as an antiviral that inhibits viral gene expression and/or viral genome replication. An EGFR signaling inhibitor may not need to completely eliminate viral gene expression to provide therapeutic effects; they may only need to reduce viral gene expression enough to restore expression of tumor suppressor proteins p53 and pRB, which in turn may direct the cells to die.

Thus, in one aspect, the invention provides a method of providing treatment to a subject exhibiting at least one symptom or clinical sign of HPV infection a composition that includes an EGFR signaling inhibitor in an amount effective to ameliorate at least one symptom or clinical sign of HPV infection. Suitable EGFR signaling inhibitors can include a compound or a composition that reduces EGFR-dependent signaling, regardless of whether the compound or composition directly binds to EGFR or a ligand of EGFR. An EGFR inhibitor may be, for example, an EGFR antagonist, a compound or composition that inhibits the EGFR tyrosine kinase domain, and/or interferes with AKT-induced, PKC-induced, and/or MAPK-induced expression.

The methods described herein may be employed to treat any condition resulting from HPV infection. Such conditions can include, for example, certain cancers such as cervical cancer, oral cancer, and squamous cell carcinoma of the head and neck (SCCHN). Conditions resulting from HPV infection also can include, howeever, non-malignant conditions such as, for example, genital warts, plantar warts, common warts, filiform warts, flat warts, respiratory papillomatosis, and cervical dysplasia.

Accordingly, ameliorating at least one symptom or clinincal sign of HPV infection can include reducing the number, frequency, and or size of at least one HPV-induced neoplasia whether the neoplasia is malignant or non-malignant. For example, ameliorating at least one symptom or clinincal sign of HPV infection can include reducing the size of an HPV-induced tumor, reducing the number of HPV-induced tumors, reducing the size of an HPV-induced non-malignant neoplasia (e.g., a wart), or the number of HPV-induced non-malignant neoplasias. Alternative symptoms and clinincal signs of HPV infection are well characterized and mey be ameliorated to any extent using the methods described herein.

Inhibition of activity—whether direct EGFR activity, tyrosine kinase activity, MAPK activity, PKC activity, or AKT activity—can be quantitatively measured and described as a percentage of the functional activity of a comparable control, e.g., in vitro or in vivo activity in the absence of the inhibitor. Combinations of inhibitors targeting more than one of these EGFR signal arms (AKT, Ras/MAPK, PKC) may result in compounded HPV inhibition.

Inhibition can be expressed either as a percentage decrease in activity or, alternatively, as the percentage of remaining activity compared to a comparable. Thus, inhibition can be expressed as a decrease in activity that is at least 5% (e.g., a decrease of 5 units of activity compared to a control exhibiting 100 units of activity), at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% of the activity of a suitable control. Alternatively, inhibition can be expressed as remaining activity that is no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, or no more than 99% of the activity of a suitable control.

An EGFR antagonist can include any compound or composition that binds to the extracellular domain of EGFR and results in a level of EGFR cell signaling that is less than the EGFR-dependent cell signaling induced by any natural agonist of EGFR. Natural EGFR ligands include, for example, epidermal growth factor (EGF), transforming growth factor α (TGFα), heparin-binding EGF-like growth factor (HB-EGF), amphiregulin, betacellulin, epigen, and epiregulin.

Thus, suitable EGFR signaling inhibitors can include monoclonal antibodies that bind to

EGFR such as, for example, therapeutic monoclonal antibodies cetuximab, panitumumab, pertuzumab, zalutumumab, nimotuzumab, or matuzumab. In some embodiments, a suitable EGFR signaling inhibitor can include a small molecule tyrosine kinase inhibitor such as, for example, gefitinib, erlotinib, lapatinib, genistein, AG1478 (tyrphostin), PD168393, or GW572016. In some embodiments, a suitable EGFR signaling inhibitor can include a MAPK inhibitor such as, for example, PD98059, U0126, or SL327. In some embodiments, a suitable EGFR signaling inhibitor can include an AKT inhibitor such as, for example, LY294002 (2-morpholin-4-yl-8-phenylchromen-4-one), the farnesyl transferase inhibitor R115777 (tipifarnib, e.g., ZARNESTRA, Johnson & Johnson Research & Development, LLC, Raritan, N.J.), the Raf antisense oligonucleotide ISIS 5132, the Raf inhibitor Bay 43-9006 (sorafinib, e.g., NEXAVAR, Onyx Pharmaceuticasls, Inc., South San Francisco, Calif. and Bayer HealthCare Pharmaceuticals, Inc., Berlin, Germany), the MEK inhibitor CI-1040 (PD184352, Pfizer, Inc., New York, N.Y.), the mTOR inhibitor CCI-779 (temsirolimus, e.g., TORISEL, Wyeth Pharmaceuticals, Collegeville, Pa.), the mTOR inhibitor RAD001 (everlimus, e.g., ZORTRESS, Novartis Pharmaceuticals Corp., East Hanover, N.J.), the topoisomerase II inhibitor C1311 (e.g., SYMADEX, Xanthus Pharmaceuticals, Inc., Cambridge, Mass.), or the heat shock protein 90 (Hsp90) inhibitor tanespimycin (Bristol-Myers Squibb, New York, N.Y.). Other suitable EGFR signaling inhibitors can include, for example, enzastaurin, quercetin, or PKC412 (midostaurin).

In some embodiments, the EGFR signaling inhibitor may be co-administered with at least one additional therapeutic composition to provide combination therapy. The additional therapeutic composition can include one or more compounds that can supplement the therapy provided by the EGFR signaling inhibitor. Thus, the additional therapeutic composition may possess anti-tumor activity, anti-viral activity, anti-inflammatory activity, immunostimulatory activity, etc.

One or more EGFR signaling inhibitors may be formulated in a composition along with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with the EGFR signaling inhibitor without causing any undesirable biological effects or interacting in a deleterious manner with any of the other components of the pharmaceutical composition in which it is contained.

One or more EGFR signaling inhibitors may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). It is foreseen that a composition can be administered to a mucosal surface, such as by administration to, for example, the nasal or respiratory mucosa (e.g., by spray or aerosol). A composition also can be administered via a sustained or delayed release.

Many HPV infections occur at epithelial surfaces, many of which are accessible to topical treatment. Thus, in some embodiments, one or more EGFR signaling inhibitors can be administered topically, either alone or in addition to a systemic treatment that may or may not include an EGFR signaling inhibitor.

A formulation may be conveniently presented in unit dosage form and may be prepared by methods well known in the art of pharmacy. Methods of preparing a composition with a pharmaceutically acceptable carrier include the step of bringing the one or more EGFR signaling inhibitors into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

One or more EGFR signaling inhibitors may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

The amount of the one or more EGFR signaling inhibitors administered can vary depending on various factors including, but not limited to, the specific EGFR signaling inhibitor or inhibitors being administered, the weight, physical condition, and/or age of the subject, and/or the route of administration. Thus, the absolute weight of EGFR signaling inhibitor included in a given unit dosage form can vary widely, and depends upon factors such as the species, age, weight and physical condition of the subject, as well as the method of administration. Accordingly, it is not practical to set forth generally the amount that constitutes an amount of one EGFR signaling inhibitor effective for all possible applications. Those of ordinary skill in the art, however, can readily determine the appropriate amount with due consideration of such factors.

In some embodiments, the methods of the present invention include administering sufficient EGFR signaling inhibitor to provide a dose of, for example, from about 100 ng/kg to about 50 mg/kg to the subject, although in some embodiments the methods may be performed by administering EGFR signaling inhibitor in a dose outside this range. In some of these embodiments, the method includes administering sufficient EGFR signaling inhibitor to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the methods of the present invention may include administering sufficient EGFR signaling inhibitor to provide a dose of, for example, from about 0.01 mg/m² to about 1000 mg/m² such as, for example, a dose of about 500 mg/m². In some cases, an EGFR signaling inhibitor can be administered at one initial dose of, for example, 400 mg/m², then followed by subsequent lesser maintenance doses such as, for example, 250 mg/m²/week.

In some embodiments, EGFR signaling inhibitor may be administered, for example, from a single dose to multiple doses per week, although in some embodiments the methods of the present invention may be performed by administering EGFR signaling inhibitor at a frequency outside this range. In certain embodiments, EGFR signaling inhibitor may be administered from about once every 12 weeks, once every eight weeks, once every four weeks, or once every week.

In the preceding description, particular embodiments may be described in isolation for clarity. Unless otherwise expressly specified that the features of a particular embodiment are incompatible with the features of another embodiment, certain embodiment can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Quantification of EGFR Levels in HPV 16-Negative and HPV 16-Positive Keratinocyte Cell Lines

Keratinocyte cell lines were serum starved prior to treatment for 24 hours, then treated with 5 μM EGF for 0 minutes, 5 minutes, 15 minutes or 24 hours, then were lysed and collected. The lysates were immunoblotted for: EGFR and GAPDH for loading control. Results are shown in FIG. 3.

### Example 2

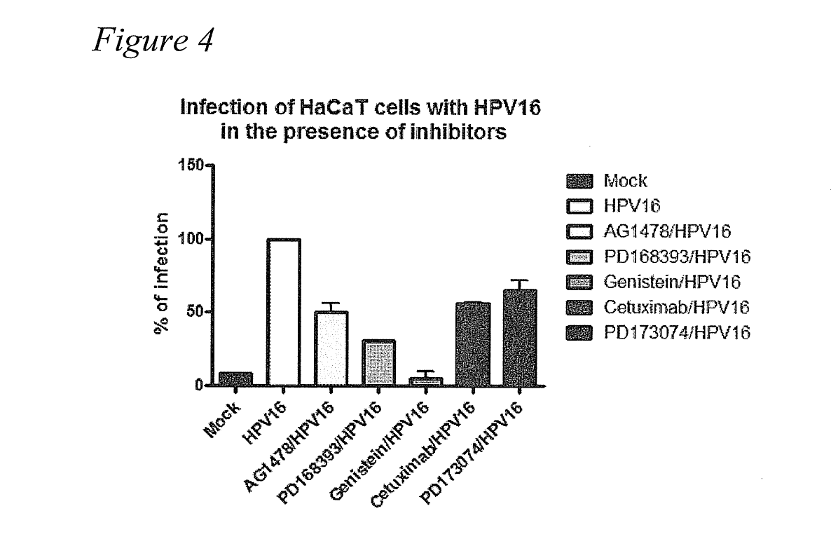

HaCaT cells, a human keratinocyte cell line, were pre-treated 30 minutes with 1 mM AG1478, a reversible EGFR inhibitor, 100 nM PD168393, an irreversible EGFR inhibitor, 100 mM genistein, 50 nmol cetuximab, or 100 nM PD173074, a keratinocyte growth factor receptor antagonist. Cells were exposed to HPV16 or HPV31 PsV at 100 vge/cell for one hour at 4° C., then shifted to 37° C. in the presence of inhibitors for 24 hours, at which time they were analyzed for luciferase reporter gene expression quantification. Raw data were normalized to total protein content and compared to untreated virus infections, which were set to 100%. Resutls are shown in FIG. 4.

### Example 3

### HPV Infection Activates the EGFR Signaling Pathways

SG3 cells (HPV16[+]) and NIKS cells (HPV[−] cells) were serum starved for 8 hours prior to mock (M) treatment, treatment with diluent in serum free medium (SFM) and 10 ng/mL EGF (Ø), cetuximab (C, 100 μg/mL), or MEK inhibitor PD98059 (P, 25 μM) before and during exposure to 10 ng/mL EGF in SFM for five minutes or 15 minutes. Mock=No EGF. Results are shown in FIG. 5.

### Example 4

### HPV16 Exposure Causes Activation of AKT and mTOR

Human keratinocytes were serum-starved for four hours, then mock exposed or exposed to EGF (5 ng/mL), HPV16 (100 vge/cell) for the times indicated. (A). After serum-starvation, cells were treated with inhibitors (1 μM AG1478 [EGFR inhibitor], 25 μM LY294002 [PI3K inhibitor] and 1 μM wortmannin [PI3K inhibitor]) or DMSO for one hour at 37° C. Following incubation with inhibitors, cells were treated with 100 vge/cell HPV 16 PsV for 15 minutes at 37° C. in the presence of inhibitors. Cells were washed with ice cold PBS and lysed with radioimmuneprecipitation assay (RIPA) buffer (Sigma-Aldrich, St. Louis, Mo.), clarified, solubilized in loading buffer and fractionated by SDS-PAGE. Immunoblot analysis was performed using anti-pAKT (Ser 473) and pAKT (Thr 308) antibodies. Data are representative of three independent assays. (B) The cell lysates were analyzed by SDS-PAGE and immunoblot for p-PTEN (Ser 380), an inhibitor of ATK/mTOR. (C) Cell lysates were analyzed for p-mTOR (Ser 2448 and Ser 2481). Each was target phosphorylated in response to HPV exposure. The results show that EGFR activation also leads to AKT/mTOR activation in keratinocytes. Results are in FIG. 6.

### Example 5

### EGFR Inhibitors Cause Decreased Proliferation in HPV[+] Cells

Cells were grown for seven days in fibroblast conditioned E media containing (0 μg/mL [M], 50 μg/mL, 100 μg/mL, or 200 μg/mL cetuximab or 12.5 μM, 25 μM, or 50 μM PD98059, as indicated by the wedges. The boxes indicate the same concentrations used in the FIG. 5 and FIG. 8. MTT assays were performed to assess cell viability. Error bars represent SEM (N=3). Results are shown in FIG. 7.

### Example 6

### EGFR Inhibitors Cause Decreased HPV 16 Transcription and Viral Genome Levels

HPV[+] HFK cells were incubated in fibroblast conditioned media containing 100 μg/mL cetuximab or 25 μM PD98059. Total RNA harvested at 24 hours, 48 hours, 72 hours, or 96 hours was subjected to RT and then to qPCR for β-actin and HPV16 E1̂E4 quantification. Statistically significant decreases in HPV16 E1̂E4 transcript levels in response to treatments are indicated. Data are the result of two separate experiments and are shown in FIG. 8.

HPV[+] HFK cells were incubated in fibroblast conditioned media containing 100 μg/mL cetuximab (M=Media only) or 25 μM PD98059 (Ø=Media+DMSO). Total DNA harvested at two days, three days, four days, five days, or six days after treatment was subjected to qPCR (SYBR Green I kit on the BioRad iCycler) for HPV16 LCR quantification. Statistically significant decreases in HPV16 genome levels are indicated. Data are the result of two separate experiments and are shown in FIG. 9. Total RNAs were extracted from cells using TRIzol (Invitrogen), and processed with the

TURBO DNA-free Kit (Ambion) to remove co-purifying viral and cellular DNA. Reverse transcription (RT) of total RNAs (0.2-0.5 μg) was performed using random hexamer primers and ABI reagents (Applied Biosystems). For quantitative PCR (qPCR), 2 μl of each cDNA or total DNA was analyzed in triplicate using the SYBR Green I kit on the iCycler (Bio-Rad Laboratories, Inc., Hercules, Calif.).

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
administering to a subject exhibiting at least one symptom or clinical sign of HPV infection a composition that includes an EGFR signaling inhibitor in an amount effective to ameliorate at least the exhibiting at least one symptom or clinical sign of HPV infection.

2. The method of claim 1 wherein the at least one symptom or clinical sign of HPV infection comprises at least one symptom or clinical sign of a cancer.

3. The method of claim 2 wherein the cancer comprises cervical cancer or oral cancer.

4. The method of claim 1 wherein the EGFR signaling inhibitor comprises an antagonist of EGFR.

5. The method of claim 4 wherein the antagonist of EGFR comprises a monoclonal antibody.

6. The method of claim 1 wherein the EGFR signaling inhibitor comprises a tyrosine kinase inhibitor.

7. The method of claim 1 wherein the EGFR inhibitor comprises a MAPK inhibitor.

8. The method of claim 1 wherein the EGFR inhibitor comprises an ERK1/2 inhibitor.

9. The method of claim 1 wherein the EGFR inhibitor comprises a PKC inhibitor.

10. The method of claim 1 wherein the EGFR inhibitor comprises an AKT inhibitor.

11. The method of claim 1 wherein the EGFR inhibitor comprises an mTOR inhibitor.
